(19) United States
(12) Patent Application Publication
(10) Pub. No.: US 2021/0196827 A1
(43) Pub. Date: Jul. 1, 2021

(54) FUNCTIONAL NUCLEIC ACID HAVING NUCLEOSIDE ANALOG DRUG INTEGRATED INTO SKELETON, DERIVATIVE AND USE THEREOF

(71) Applicant: SHANGHAI JIAOTONG UNIVERSITY, Shanghai (CN)

(72) Inventors: Chuan ZHANG, Shanghai (CN); Yuan MA, Shanghai (CN); Quanbing MOU, Shanghai (CN); Xinyuan ZHU, Shanghai (CN)

(21) Appl. No.: 17/057,525

(22) PCT Filed: Dec. 14, 2018
(86) PCT No.: PCT/CN2018/121076

§ 371 Date: Nov. 20, 2020

(30) Foreign Application Priority Data
May 21, 2018 (CN) 201810489585.6

(51) Int. Cl. A61K 47/26; C12N 15/113

(52) U.S. Cl. CPC A61K 47/26; C12N 15/113

(57) ABSTRACT

The application discloses a functional nucleic acid having nucleoside analog drug integrated into skeleton, a derivative, and preparation methods thereof wherein the derivative is obtained by conjugating or self-assembling the functional nucleic acid having nucleoside analog drug integrated into skeleton with one of a polymer, a hydrophobic molecule, and a transfection reagent. Compared with the prior art, the functional nucleic acid having nucleoside analog drug integrated into skeleton and the derivative thereof can efficiently enter cells and be used to regulate genes; subsequently, the functional nucleic acid having nucleoside analog drug integrated into skeleton can be degraded by nuclease and release active ingredient of the nucleoside analog drug, thus playing a role in chemotherapy. Hence, the functional nucleic acid having nucleoside analog drug integrated into skeleton and the derivative thereof can simply and efficiently realize a combination therapy of gene therapy and chemotherapy, and a complex synthesis procedure is avoided.

### CROSS REFERENCE TO RELATED APPLICATION

This application claims the priority of Chinese Patent Application No. 201810489585.6 entitled “Functional nucleic acid having nucleoside analog drug integrated into skeleton, derivative and use thereof” filed with China National Intellectual Property Administration on May 21, 2018, which is incorporated herein by reference in its entirety.

### TECHNICAL FIELD

The disclosure belongs to the technical field of biological medicine, especially relating to a functional nucleic acid having nucleoside analog drug integrated into skeleton, derivative, preparation method and use thereof.

### BACKGROUND ART

During chemotherapy, drug resistance of tumor cells is a great challenge in the field of cancer treatment (Nat. Rev. Cancer 2013, 13, 714-726.).

There are many factors that lead to drug resistance of tumor cells, such as enhanced anti-apoptotic ability, increased drug pumping rate, etc. (Nat. Rev. Cancer 2012, 12, 494-501.). For example, in order to avoid the injury of chemotherapeutic drugs, tumor cells will over-express anti-apoptotic proteins during chemotherapy, thus inhibiting the anti-tumor activity of chemotherapeutic drugs. In addition, tumor cells may also up-regulate the expression of drug transporters, which have the ability to pump chemotherapeutic drugs out of the cells. Therefore, the up-regulation of transporters will reduce the drug concentration in tumor cells, thereby reducing the efficacy.

At present, in order to solve the problem of tumor drug resistance, the combination of gene therapy and chemotherapy is mainly used. Firstly, gene therapy is used to reverse the expression of drug resistance-related genes, so as to restore the sensitivity of tumor cells to chemotherapeutic drugs, thereby prompting chemotherapeutic drugs to effectively inhibit tumor proliferation (Proc. Natl. Acad. Sci. U.S.A. 2010, 107, 10737-10742.).

However, there are certain problems when gene drugs and chemotherapeutic drugs are used in direct combination, for example, short blood half-life of both drugs, instability of genes in vivo and low efficiency of endocytosis, poor tumor targeting, and potential toxic side effects, etc. (Nat. Rev. Genet. 2014, 15, 541-555.).

In order to improve the above problems, in recent years, scientific researchers have tried to use nano-drug delivery systems for the synergistic delivery of gene drugs and chemotherapeutic drugs (Nat. Nanotechnol. 2011, 6, 658-667.), considering that the nano-delivery system has the advantages of long half-life, good stability in vivo, fast cellular uptake rate, and low side effects (Nat. Biotechnol. 2016, 34, 414-418.). So far, researchers have developed many kinds of nano-delivery vehicles, such as micelles (J. Am. Chem. Soc. 2016, 138, 10834-10837.), liposomes (Proc. Natl. Acad. Sci. USA 2010, 107, 10737-10742.), and nucleic acid assembly (J. Am. Chem. Soc. 2013, 135, 18644-18650.), etc.

Although these nanocarrier-based synergistic delivery systems of gene drug and chemotherapeutic drug have been carefully designed, these nano synergistic delivery systems still face many challenges due to the great differences in physicochemical properties between gene drugs and chemotherapeutic drugs, including:

(1) The hydrophilicity and hydrophobicity of gene drugs and chemotherapeutic drugs are significantly different from each other, so that it is difficult to design an ideal nanocarrier to efficiently encapsulate both substances at the same time;

(2) It is difficult to precisely control the drug-loading and the ratio of gene drugs and chemotherapeutic drugs in the carrier;

(3) It is difficult to precisely control the time sequence of gene drugs release and chemotherapeutic drugs release.

The above difficulties will limit the use of gene-chemotherapeutic combination therapy in the treatment of drug-resistant tumors.

### SUMMARY OF THE INVENTION

The first purpose of the disclosure is to provide a functional nucleic acid having nucleoside analog drug integrated into skeleton to realize the combined delivery of gene drugs and chemotherapeutic drugs, so as to solve the problems existing in the combined delivery technology, including: (1) The hydrophilicity and hydrophobicity of gene drugs and chemotherapeutic drugs are significantly different from each other, so that it is difficult to design an ideal nanocarrier to efficiently encapsulate both substances at the same time; (2) It is difficult to precisely control the drug-loading and the ratio of gene drugs and chemotherapeutic drugs in the carrier; (3) It is difficult to precisely control the time sequence of gene drugs release and chemotherapeutic drugs release.

The second purpose of the disclosure is to provide a method for preparing the functional nucleic acid having nucleoside analog drug integrated into skeleton.

The third purpose of the disclosure is to provide a derivative based on the above-mentioned functional nucleic acid having nucleoside analog drug integrated into skeleton to realize efficient delivery of the functional nucleic acid having nucleoside analog drug integrated into skeleton in cells.

The fourth purpose of the disclosure is to provide a method for preparing the derivative based on the functional nucleic acid having nucleoside analog drug integrated into skeleton to realize efficient delivery of the functional nucleic acid having nucleoside analog drug integrated into skeleton in cells.

The fifth purpose of the disclosure is to provide a use of the functional nucleic acid having nucleoside analog drug integrated into skeleton and derivative thereof for preparing nucleic acid drugs and chemotherapeutic drugs for treating diseases based on the combination of gene therapy and chemotherapy.

The technical schemes of the disclosure are as follows:

A functional nucleic acid having nucleoside analog drug integrated into skeleton, wherein the nucleoside analog drug is integrated into the skeleton of the functional nucleic acid by replacing natural nucleotides in the functional nucleic acid after chemical modification.

In some embodiments, the chemical modification method of the nucleoside analog drug may include that the nucleoside analog drug is modified into its phosphoramidite derivative or triphosphate derivative.

In some embodiments, the nucleoside analog drug is integrated into the skeleton of the functional nucleic acid by a solid-phase synthesis or an in vitro enzyme transcription technology or a PCR amplification after the nucleoside analog drug is chemically modified.

In some embodiments, the ratio of genes to drugs can be adjusted accurately by adjusting the number of natural nucleotides replaced by drugs, which solves the problems that the drug-loading and ratio of gene drugs and chemotherapeutic drugs in the carrier are difficult to precisely control.

In some embodiments, the functional nucleic acid having nucleoside analog drug integrated into skeleton is selected from antisense oligonucleotides (antisense DNA), small interfering RNA (siRNA), messenger RNA (mRNA), microRNA (micro RNA), long non-coding RNA (lncRNA), small hairpin RNA (shRNA), guide RNA for gene editing (sgRNA, etc.), and circular RNA (circRNA), but not limited to these functional nucleic acids.

In some embodiments, the functional nucleic acid having nucleoside analog drug integrated into skeleton, wherein the nucleoside analog drug is selected from the following drugs:

Purine analogs, which may be Mercaptopurine, Tioguanine, Azathioprine or 8-Azaguanin;

Guanosine analogs, which may be Nelarabine or Forodesine;

Cytidine analogs, which may be Cytarabine, Ancitabine, Gemcitabine, Enocitabine, 5-Azacytidine or Decitabine;

Adenosine analogs, which may be Fludarabine, Cladribine, Clofarabine or Acadesine;

Uridine analogs, which may be Fluorouracil, Carmofur, Tegafur, 5′-Deoxy-5-fluorouridine, Capecitabine or Floxuridine.

However, the technical schemes of the disclosure are not limited to the above-mentioned nucleoside analog drugs.

The disclosure also provides a method for preparing the above-mentioned functional nucleic acid having nucleoside analog drug integrated into skeleton, which includes one of the following methods:

using the phosphoramidite monomer of the nucleoside analog drug, preparing a functional nucleic acid having nucleoside analog drug integrated into skeleton by solid-phase synthesis method; and

using the triphosphate monomer of the nucleoside analog drugs, preparing a functional nucleic acid having nucleoside analog drug integrated into skeleton by in vitro enzyme transcription method or PCR amplification. In an embodiment, the method comprising the following steps:

inputting the sequence of the functional nucleic acid to be synthesized into a solid-phase synthesizer, and preparing a functional nucleic acid having nucleoside analog drug integrated into skeleton on the surface of ordinary CPG or an amino-modified CPG; and

reacting a mixed solution of RNA polymerase, template DNA, RNA nucleoside triphosphate monomer, triphosphate monomer of nucleoside analog drug, and reaction buffer at 37° C. to prepare a functional nucleic acid having nucleoside analog drug integrated into skeleton;

reacting a mixed solution of DNA polymerase, template DNA, DNA primer, DNA nucleoside triphosphate monomer, triphosphate monomer of nucleoside analog drug, and reaction buffer at 37° C. to prepare a functional nucleic acid having nucleoside analog drug integrated into skeleton.

The above-mentioned functional nucleic acids having nucleoside analog drug integrated into skeleton of the disclosure may be prepared into various derivatives, including but not limited to:

1. A functional derivative that formed by combining the functional nucleic acid having nucleoside analog drug integrated into skeleton with a molecular targeting group, such as a nucleic acid aptamer, a targeting polypeptide, a targeting small molecule etc.;

2. The functional nucleic acid having nucleoside analog drug integrated into skeleton may also be modified by a polymer or a hydrophobic molecule to obtain a derivative and a self-assembled nanostructure thereof, and

3. The functional nucleic acid having nucleoside analog drug integrated into skeleton may also be self-assembled with a transfection reagent to obtain a derivative of composite nanostructure; and

4. The functional nucleic acid having nucleoside analog drug integrated into skeleton is modified by a polymer or a hydrophobic molecule to obtain a functional nucleic acid derivative, and the functional nucleic acid derivative may be self-assembled with a transfection reagent to obtain a derivative of composite nanostructure.

The disclosure further provides a derivative of the above-mentioned functional nucleic acid having nucleoside analog drug integrated into skeleton, which includes one of the followings:

a functionalized derivative formed by combining the functional nucleic acid having nucleoside analog drug integrated into skeleton with a molecular targeting group; and

a derivative and a self-assembled nanostructure thereof obtained by modifying the functional nucleic acid having nucleoside analog drug integrated into skeleton with a polymer or a hydrophobic molecule; and

a composite nanostructure obtained by self-assembly of the functional nucleic acid having nucleoside analog drug integrated into skeleton and transfection reagent; and

a composite nanostructure obtained by self-assembly of a modified product, which was obtained by modifying the functional nucleic acid having nucleoside analog drug integrated into skeleton with a polymer or a hydrophobic molecule, and a transfection reagent.

In some embodiments, the polymer is selected from polycaprolactone, polyethylene glycol, and polylactic acid-glycolic acid copolymer. But the disclosure is not limited to the above-mentioned polymers.

In some embodiments, the hydrophobic molecules are selected from the followings:

phospholipid molecules, such as phosphatidylcholine, phosphatidylethanolamine, phosphatidylserine, phosphatidylglycerol, phosphatidylinositol, diphosphatidylglycerol, sphingomyelin, glycosphingolipid;

alkane molecules, such as pentane, dodecane, octadecane;

steroid molecules, such as cholesterol, cortisol, aldosterone, testosterone, estradiol, and vitamin D. Examples are not limited thereto to modify the functional nucleic acid having nucleoside analog drug integrated into skeleton only by using the above-mentioned hydrophobic molecules.

In some embodiments, the transfection reagents are selected from polyethyleneimine, polylysine, chitosan, Lipofectamine™ transfection reagent, Lipofectamine™ 2000 transfection reagent, Lipofectamine™ 3000 transfection reagent, Lipofectamine™ RNAiMAX transfection reagent, gold nanoparticles, ferroferric oxide nanoparticles, and silica nanoparticles. But it is not limited to the above-mentioned cationic polymers, cationic liposomes, inorganic nanoparticles, and other transfection reagents that can be used for functional nucleic acid loading and transfection.

A method for preparing the functional nucleic acid having nucleoside analog drug integrated into skeleton provided by the disclosure mainly includes:

using targeting molecules, including nucleic acid aptamers, targeted polypeptides, targeted small molecules and the like to modify the functional nucleic acid, obtaining the derivatives of the functional nucleic acid having nucleoside analog drug integrated into skeleton, and

using polymers to modify the functional nucleic acid, obtaining the derivatives of the functional nucleic acid having nucleoside analog drug integrated into skeleton; wherein the polymers are selected from (but not limited to) polycaprolactone, polyethylene glycol, and polylactic acid-glycolic acid copolymer; and

using hydrophobic molecules to modify the functional nucleic acid, obtaining the derivatives of the functional nucleic acid having nucleoside analog drug integrated into skeleton; wherein the hydrophobic molecules are selected from (but not limited to) phospholipid molecules, for example one of phosphatidylcholine, phosphatidylethanolamine, phosphatidylserine, phosphatidylglycerol, phosphatidylinositol, diphosphatidylglycerol, sphingomyelin, glycosphingolipid; alkane molecules, for example, one of pentane, dodecane, octadecane, etc.; steroid molecules, for example, one of cholesterol, cortisol, aldosterone, testosterone, estradiol and vitamin D; and

self-assembling the functional nucleic acid having nucleoside analog drug integrated into skeleton with the transfection reagent to obtain a derivative of composite nanostructure; or self-assembling the functional nucleic acid having nucleoside analog drug integrated into skeleton or modified product thereof, which is modified by a polymer or a hydrophobic molecule and a transfection reagent, with the transfection reagent to obtain a derivative of composite nanostructure, wherein the transfection reagents are selected from polyethyleneimine, polylysine, chitosan, Lipofectamine™ transfection reagent, Lipofectamine™ 2000 transfection reagent, Lipofectamine™ 3000 transfection reagent, Lipofectamine™ RNAiMAX transfection reagent, gold nanoparticles, ferroferric oxide nanoparticles, and silica nanoparticles, etc. Examples are not limited to the above-mentioned cationic polymers, cationic liposomes, inorganic nanoparticles, and other transfection reagents that can be used for functional nucleic acid loading and transfection.

In a preferred embodiment, in the method for preparing the derivatives of the functional nucleic acid having nucleoside analog drug integrated into skeleton, the method of modifying with polymers includes the following steps:

providing a degradable polymer with an azide group at the terminus synthesized by ring-opening polymerization, and then carrying out a copper-free catalytic click(Click) reaction between the degradable polymers and the functional nucleic acid having nucleoside analog drug integrated into skeleton modified by diphenyl-cyclooctyne, obtaining a conjugate of the degradable polymer-functional nucleic acid having nucleoside analog drug integrated into skeleton, as one of the derivatives of the functional nucleic acid having nucleoside analog drug integrated into skeleton.

The disclosure also provides a method for preparing an aqueous solution of the above-mentioned derivatives of the functional nucleic acid having nucleoside analog drug integrated into skeleton, which includes the following steps:

dissolving the conjugate of the degradable polymer-functional nucleic acid having nucleoside analog drug integrated into skeleton in dimethyl sulfoxide (DMSO), and then dialyzing the solution in ultrapure water for 24 hours, obtaining an aqueous solution of the conjugate of the degradable polymer-functional nucleic acid having nucleoside analog drug integrated into skeleton, as the aqueous solution of the derivatives of the functional nucleic acid having nucleoside analog drug integrated into skeleton.

The disclosure also discloses the use of the functional nucleic acid having nucleoside analog drug integrated into skeleton and derivatives thereof for preparing nucleic acid drugs and chemotherapeutic drugs for treating diseases based on the combination of gene therapy and chemotherapy.

Compared with the prior art, the beneficial effects of the disclosure are as follows:

First, the functional nucleic acid having nucleoside analog drug integrated into skeleton and derivatives thereof in the disclosure are able to achieve precise adjustment of the ratio of genes to drugs, and particularly can be achieved by adjusting the number of natural nucleotides replaced by drugs;

Second, the functional nucleic acid having nucleoside analog drug integrated into skeleton and derivatives thereof in the disclosure are able to make the genes and drugs play their roles procedurally in sequence and can maximize the effect of synergistic therapy;

Third, a vector is successfully designed in the disclosure, which can efficiently encapsulate or assemble both gene drugs and chemotherapeutic drugs at the same time.

The implementation of any product of the disclosure does not necessarily need to achieve all the advantages described above at the same time.

### BRIEF DESCRIPTION OF THE DRAWINGS

FIG. 1 shows the changes in tumor size during therapy of the Bcl-2 antisense oligonucleotide (F-Bcl-2 ASO) of skeleton integrated floxuridine in Example 1 of the disclosure on the drug-resistant liver orthotopic transplantation tumor in tumor-bearing nude mice;

FIG. 2 is a picture showing a tumor-bearing liver after therapy of the Bcl-2 antisense oligonucleotide (F-Bcl-2 ASO) of skeleton integrated floxuridine in Example 1 of the disclosure on the drug-resistant liver orthotopic transplantation tumor in tumor-bearing nude mice;

FIG. 3 shows that after therapy of the Bcl-2 antisense oligonucleotide (F-Bcl-2 ASO) of skeleton integrated floxuridine in Example 1 of the disclosure is delivered on the drug-resistant liver orthotopic transplantation tumor in tumor-bearing nude mice, the expression amount of drug resistance-related proteins in drug-resistant tumors is down-regulated;

FIG. 4 shows that the Bcl-2/xL antisense oligonucleotide (F-Bcl-2/xL ASO) of skeleton integrated floxuridine in Example 2 of the disclosure down-regulates the expression amount of drug resistance-related proteins in drug-resistant cells BEL-7402;

FIG. 5 shows a synthetic route of the spherical nucleic acid SNA (F-Bcl-2 ASO) constructed by Bcl-2 antisense oligonucleotide of skeleton integrated fluorouridine in Example 3 of the disclosure;

FIG. 6 shows a 20% denatured polyacrylamide gel electrophoresis spectrum of the Bcl-2 antisense oligonucleotide (F-Bcl-2 ASO-DBCO) of skeleton integrated floxuridine modified by dibenzocyclooctyne (DBCO) in Example 3 of the disclosure;

FIG. 7 shows a synthetic route of polymer 1 (N₃-PEG-b-PCL₂₈) in Example 3 of the disclosure;

FIG. 8 shows a ¹H NMR spectrum of polymer 1 (N₃-PEG-b-PCL₂₈) in Example 3 of the disclosure;

FIG. 9 shows a synthetic route of click reaction conjugation of polymer 1 (N₃-PEG-b-PCL₂₈) and the Bcl-2 antisense oligonucleotide (F-Bcl-2 ASO-DBCO) of skeleton integrated floxuridine modified by DBCO in Example 3 of the disclosure;

FIG. 10 shows a 1% non-denaturing agarose gel electrophoresis spectrum of the spherical nucleic acid SNA (F-Bcl-2 ASO) constructed by Bcl-2 antisense oligonucleotide of skeleton integrated fluorouridine in Example 3 of the disclosure;

FIG. 11 shows a dynamic light scattering diagram of the spherical nucleic acid SNA (F-Bcl-2 ASO) constructed by Bcl-2 antisense oligonucleotide of skeleton integrated fluorouridine in Example 3 of the disclosure;

FIG. 12 shows a transmission electron micrograph of the spherical nucleic acid SNA (F-Bcl-2 ASO) constructed by Bcl-2 antisense oligonucleotide of skeleton integrated fluorouridine in Example 3 of the disclosure;

FIG. 13 shows the changes in tumor size during therapy of the spherical nucleic acid SNA (F-Bcl-2 ASO) constructed by Bcl-2 antisense oligonucleotide of skeleton integrated fluorouridine in Example 3 of the disclosure on the drug-resistant liver orthotopic transplantation tumor in tumor-bearing nude mice;

FIG. 14 is a picture showing a tumor-bearing liver after therapy of the spherical nucleic acid SNA (F-Bcl-2 ASO) constructed by Bcl-2 antisense oligonucleotide of skeleton integrated fluorouridine in Example 3 of the disclosure on the drug-resistant liver orthotopic transplantation tumor in tumor-bearing nude mice;

FIG. 15 shows that after therapy of the spherical nucleic acid SNA (F-Bcl-2 ASO) constructed by Bcl-2 antisense oligonucleotide of skeleton integrated fluorouridine in Example 3 of the disclosure delivered on the drug-resistant liver orthotopic transplantation tumor in tumor-bearing nude mice, the expression amount of drug resistance-related proteins in drug-resistant tumors is down-regulated;

FIG. 16 shows a 20% denatured polyacrylamide gel electrophoresis spectrum of the Bcl-2/xL antisense oligonucleotide (F-Bcl-2/xL ASO-DBCO) of skeleton integrated floxuridine modified by DBCO in Example 4 of the disclosure;

FIG. 17 shows a 1% non-denaturing agarose gel electrophoresis spectrum of the spherical nucleic acid SNA (F-Bcl-2/xL ASO) constructed by Bcl-2/xL antisense oligonucleotide of skeleton integrated fluorouridine in Example 4 of the disclosure;

FIG. 18 shows that the spherical nucleic acid SNA (F-Bcl-2/xL ASO) constructed by Bcl-2/xL antisense oligonucleotide of skeleton integrated fluorouridine in Example 4 of the disclosure down-regulates the expression amount of drug resistance-related proteins in drug-resistant cells BEL-7402.

### DETAILED DESCRIPTION OF THE EMBODIMENTS

The disclosure will be further described below in conjunction with specific embodiments. It should be understood that these embodiments are only used to illustrate the disclosure and not to limit the protection scope of the disclosure. In practical applications, improvements and adjustments made by those skilled in the art according to the present application still fall into the scope of the present application.

### Example 1 a Bcl-2 Antisense Oligonucleotide (F-Bcl-2 ASO) of Skeleton Integrated Floxuridine

1.1 Synthesis of the Bcl-2 Antisense Oligonucleotide (F-Bcl-2 ASO) of Skeleton Integrated Floxuridine

The thymine (T) nucleotides in the antisense oligonucleotides were all replaced with anti-tumor drugs fluorouridine (F) in this example during the DNA solid-phase synthesis. Specifically, the phosphorous amide monomer and DNA phosphorous amide monomer of the fluorouridine drug were placed at the corresponding positions of the DNA solid-phase synthesizer, ordinary controlled pore glass (CPG) were added to the reaction column, and the 5′-CAGCGFGCGCCAFCCFFCCCAFCCFCCFCC-3′ sequence information was input, and catalytic, capping, oxidation and deprotection reagents were added, the F-Bcl-2 ASO sequence was obtained through ammonolysis, nitrogen blowing, separation and purification of preparative chromatographic, deprotection, and concentration after synthesizing the sequence containing floxuridine.

Moreover, according to the same method, the sequence of 5′-AAFACFCCGAACGFGFCACGFCCFCAC-3′ was input into a solid-phase synthesizer, so that a disordered nucleic acid (F-scramble) of skeleton integrated floxuridine was synthesized as a control.

1.2 the Efficacy of the Bcl-2 Antisense Oligonucleotide (F-Bcl-2 ASO) of Skeleton Integrated Floxuridine on the Drug-Resistant Liver Orthotopic Transplantation Tumor in Tumor-Bearing Nude Mice

In this example, the in vivo inhibitory effect of drug-resistant tumor proliferation by the Bcl-2 antisense oligonucleotide (F-Bcl-2 ASO) of skeleton integrated floxuridine was evaluated by using the model of drug-resistant liver orthotopic transplantation tumor in tumor-bearing nude mice.

Specifically, PBS and equal equivalent fluorouridine, F-Bcl-2 ASO, F-scramble (wherein the equivalent concentration of fluorouridine for injection was 5 mg/kg bw) were injected through the tail vein, magnetic resonance imaging and Siemens Inveon Research Workplace software were used to analyze and manually circle the tumor site of each layer by three-dimensional ordered subset expectation maximization (3D-OSEM), then the image was reconstructed and the tumor size was calculated. After the treatment, the tumor-bearing liver was dissected and taken out, and then the intuitive size of the drug-resistant liver orthotopic transplantation tumor was photographed and recorded.

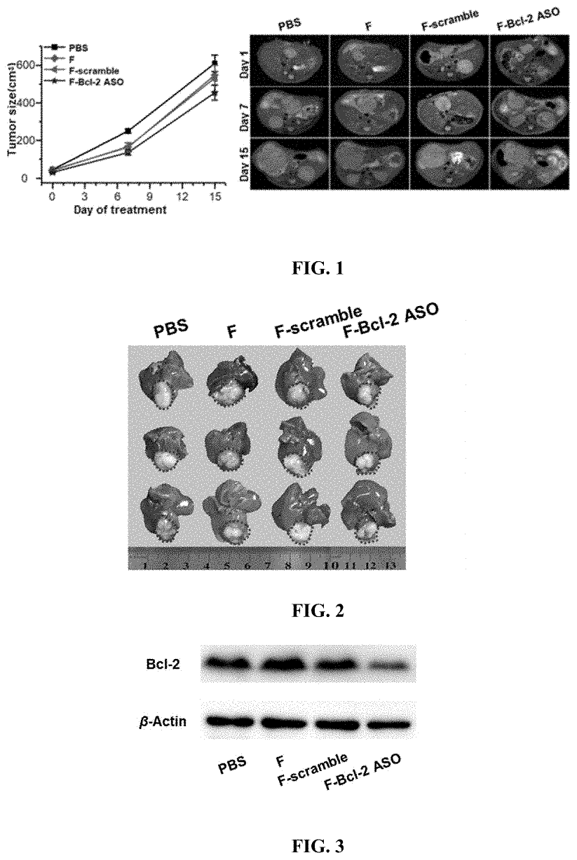

The results are shown in FIG. 1, the initial size of the drug-resistant liver transplantation tumors in each treatment group is equivalent, and the tumor size of the F-Bcl-2 ASO treatment group is smaller than that of the other control groups on the seventh and fifteenth days of treatment.

FIG. 2 shows a picture of the tumor-bearing liver taken out of the dissected nude mice after the treatment, the white part in the picture is the drug-resistant liver orthotopic transplantation tumor. It can be seen from FIG. 2 that the tumor size in the F-Bcl-2 ASO treatment group is the smallest after the treatment, while the tumor size in the remaining groups is only slightly smaller than that of the PBS control group, which means that F-Bcl-2 ASO is the most effective drug of the above drugs.

1.3 Reversing the Tumor Drug Resistance by the Bcl-2 Antisense Oligonucleotide (F-Bcl-2 ASO) of Skeleton Integrated Floxuridine Through Gene Regulation

In order to verify whether the Bcl-2 antisense oligonucleotides (F-Bcl-2 ASO) of skeleton integrated floxuridine could reverse tumor drug resistance through gene regulation, in this example, the tumor tissue was taken out after the treatment, and the total protein was extracted quickly, the expression amount of drug-resistant protein in subcutaneous drug-resistant tumors was determined by western blot analysis.

The results of the determination are shown in FIG. 3, the Bcl-2 protein band in the treatment group of F-Bcl-2 ASO is weaker than other groups, which indicates that F-Bcl-2 ASO can down-regulate the expression amount of drug-resistant proteins in tumor-bearing nude mice. Therefore, F-Bcl-2 ASO can show gene therapy effects in animals and can effectively reverse the drug resistance of drug-resistant tumors.

### Example 2 a Bcl-2/xL Antisense Oligonucleotide (F-Bcl-2/xL ASO) of Skeleton Integrated Floxuridine

2.1 Synthesis of the Bcl-2/xL Antisense Oligonucleotide (F-Bcl-2/xL ASO) of Skeleton Integrated Floxuridine

The thymine (T) nucleotides in the antisense oligonucleotides were all replaced with anti-tumor drugs fluorouridine (F) in this example during the DNA solid-phase synthesis.

Specifically, the phosphorous amide monomer and DNA phosphorous amide monomer of the fluorouridine drug were placed at the corresponding positions of the DNA solid-phase synthesizer, common controlled pore glass (CPG) were added to the reaction column, and the 5′-AAGGCAFCCCAGCCFCCGFFCCFCCFCCFA-3′ sequence information was input, and catalytic, capping, oxidation and deprotection reagents were added, the F-Bcl-2/xL ASO sequence was obtained through ammonolysis, nitrogen blowing, separation and purification of preparative chromatographic, deprotection, and concentration after synthesizing the sequence containing floxuridine.

2.2 Reversing the Tumor Drug Resistance by the Bcl-2/xL Antisense Oligonucleotide (F-Bcl-2/xL ASO) of Skeleton Integrated Floxuridine Through Gene Regulation

The Bcl-2/xL antisense oligonucleotide (F-Bcl-2/xL ASO) of skeleton integrated floxuridine were incubated with drug-resistant BEL-7402 cells for 10 hours, and then the incubation products were cultured for 48 hours after replacing by normal medium; while F-scramble and F were used as control groups, which were incubated with cells under the same condition, wherein the equivalent concentration of F was 10 μM, and drug-resistant cells without any treatment were used as a negative control group.

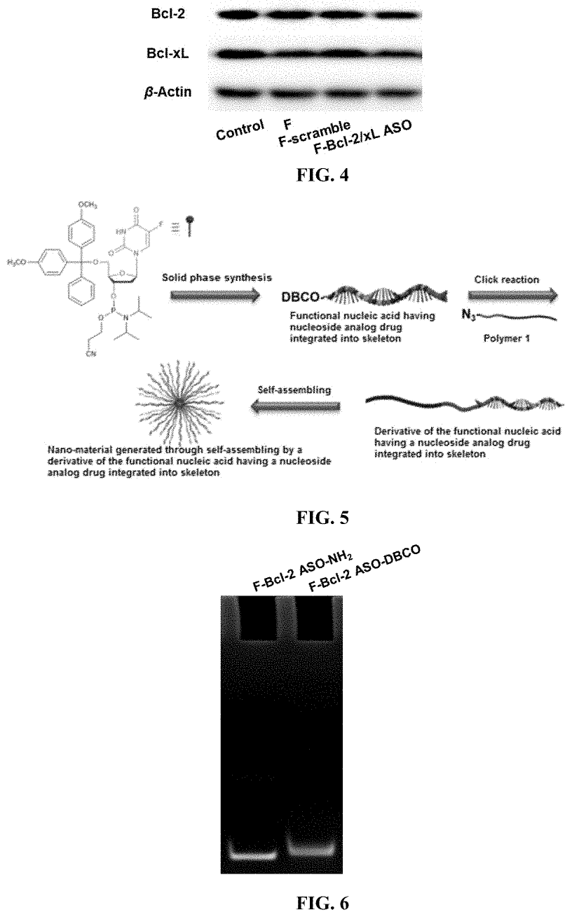

The protein expression amount of the target protein Bcl-2 in the cells was determined by Western blot analysis after extracting the total protein. The results are shown in FIG. 4, the F-Bcl-2/xL ASO can down-regulate the expression amounts of drug-resistant Bcl-2 and Bcl-xL protein after incubated with drug-resistant cells BEL-7402, whereas the expression amounts of Bcl-2 protein in the F-scramble and F treatment groups have no significant difference compared with the blank control groups. Thus, the Bcl-2/xL antisense oligonucleotide (F-Bcl-2/xL ASO) of skeleton integrated floxuridine could reverse the drug resistance of drug-resistant tumors to some extent.

### Example 3 a Spherical Nucleic Acid SNA (F-Bcl-2 ASO) Constructed by Bcl-2 Antisense Oligonucleotide of Skeleton Integrated Fluorouridine

3.1 Synthesis of the Bcl-2 Antisense Oligonucleotide (F-Bcl-2 ASO-DBCO) of Skeleton Integrated Floxuridine Modified by DBCO

The Bcl-2 antisense oligonucleotide (F-Bcl-2 ASO-NH₂) of skeleton integrated floxuridine modified by amino was prepared by using the amino-modified controlled pore glass (NH₂-CPG) when preparing Bcl-2 antisense oligonucleotide of skeleton integrated floxuridine with a solid-phase synthesis method.

The above-mentioned antisense oligonucleotide sequence was added into a DMSO mixed solution containing 30% phosphate buffer, 200 equivalents of DBCO-NHS ester was added, and the mixture was reacted at room temperature for 24 hours to obtain a Bcl-2 antisense oligonucleotide (F-Bcl-2 ASO-DBCO) of skeleton integrated floxuridine modified by DBCO (FIG. 5).

The above crude product was purified by multiple extractions with ethyl acetate, ethanol precipitation, and centrifugation.

Finally, the F-Bcl-2 ASO-DBCO was redissolved, and the DNA modification was characterized by gel electrophoresis. It can be seen from FIG. 6 that the redissolved products are presented as a single band after electrophoresis, and the products move slowly in gel electrophoresis compared with F-Bcl-2 ASO-NH₂, which proves the successful preparation of F-Bcl-2 ASO-DBCO.

3.2 Synthesis of the Polymer 1

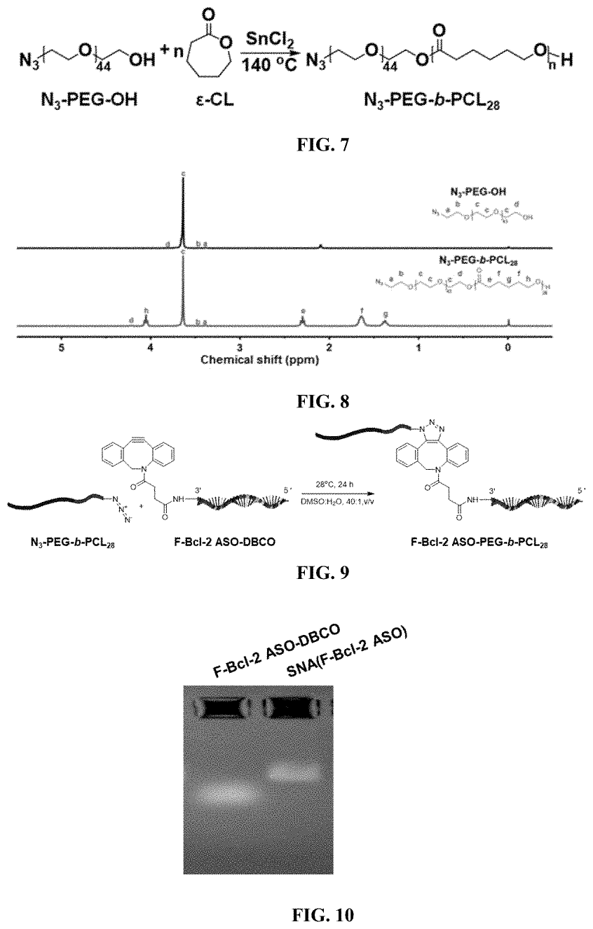

The ring-opening polymerization of ε-caprolactone was initiated by using stannous octanoate as catalyst, azide polyethylene glycol hydroxyl (N₃-PEG-OH) with a molecular weight of 2000 as an initiator to prepare the block copolymer N₃-PEG-b-PCL (FIG. 7). The specific preparation process was as follows: First, 1.0000 g (0.5 mmol) of N₃-PEG-OH and 1.7121 g (15 mmol) of anhydrous ε-caprolactone were dissolved in anhydrous toluene, followed by a catalytic amount of the stannous octanoate added through a syringe to react in a nitrogen atmosphere at 120° C. for 24 hours. After the completion of the reaction, the solvent was removed by rotary evaporation, the remaining mixture was redissolved in dichloromethane, the polymer was precipitated with ice ether, filtered and vacuum dried to obtain white powdery product polymer 1, the molecular formula was N₃-PEG-b-PCL₂ characterized by ¹H NMR spectrum.

The ¹H NMR spectrum of polymer 1 was shown in FIG. 8, the test solvent was CDCl₃, and the assignment of each proton peak was as follows: δ (ppm): 4.22 (t, 2H, —OCH₂CH₂OC(O)CH₂CH₂CH₂CH₂CH₂O—), 4.05 (t, 56H, —C(O)CH₂CH₂CH₂CH₂CH₂O—), 3.64 (s, 174H, —OCH₂CH₂O—), 2.30 (t, 56H, —C(O)CH₂CH₂CH₂CH₂CH₂O—), 1.64 (m, 112H, —C(O)CH₂CH₂CH₂CH₂CH₂O—), 1.37 (m, 56H, —C(O)CH₂CH₂CH₂CH₂CH₂O—).

3.3 Synthesis of the Spherical Nucleic Acid SNA (F-Bcl-2 ASO) Constructed by Bcl-2 Antisense Oligonucleotide of Skeleton Integrated Fluorouridine

The polymer 1 (N₃-PEG-b-PCL₂₈) and the Bcl-2 antisense oligonucleotide (F-Bcl-2 ASO-DBCO) of skeleton integrated floxuridine modified by DBCO were conjugated through classic click reaction, as shown in FIG. 9.

The synthesis process was as follows: First, N₃-PEG-b-PCL₂₈ (200 nmol) was dissolved in 1.2 mL of DMSO solution, F-Bcl-2 ASO-DBCO (400 nmol) was dissolved in 30.0 μL of water, the two solutions were shaken at 58° C. for 48 h after mixing uniformly.

After the reaction, the reaction solution was placed in a dialysis bag with a molecular weight cut-off of 10 kDa for dialysis to remove DMSO. During the dialysis process, the N₃-PEG-b-PCL₂₈ and F-Bcl-2 ASO-DBCO conjugate would gradually assemble to form a spherical nucleic acid structure SNA (F-Bcl-2 ASO) (FIG. 5).

The excess antisense oligonucleotides that did not participate in the click reaction were removed by ultrafiltration with an ultrafiltration tube with a molecular weight cut-off of 100 kDa.

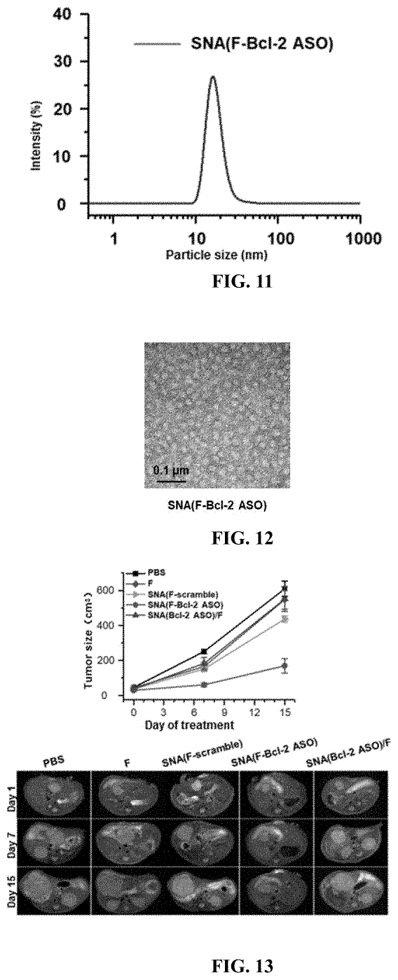

The purified SNA (F-Bcl-2 ASO) was characterized by agarose gel electrophoresis for its structure formation. The agarose gel concentration was 1%, the electrophoresis voltage was 90 V, and the gel imaging system was used for imaging after the electrophoresis was completed. The characterization results are shown in FIG. 10, the SNA (F-Bcl-2 ASO) band is located above the F-Bcl-2 ASO-DBCO band of the control group, which proves that the successful preparation of spherical nucleic acid SNA (F-Bcl-2 ASO) constructed by Bcl-2 antisense oligonucleotide of skeleton integrated fluorouridine.

In addition, according to the same method, the spherical nucleic acid SNA (F-scramble) constructed by disordered nucleic acid of skeleton integrated fluorouridine could be synthesized as a control.

The hydrated particle size of the spherical nucleic acid SNA (F-Bcl-2 ASO) constructed by Bcl-2 antisense oligonucleotide of skeleton integrated fluorouridine was characterized by dynamic light scattering experiments, and the test results were shown in FIG. 11. It can be seen that the hydrated particle size of SNA (F-Bcl-2 ASO) was 17.5 nm. In addition, the experimental results of transmission electron microscopy shown in FIG. 12 confirm that the morphology of SNA (F-Bcl-2 ASO) was spherical.

3.4 the Therapeutic Effect of the Spherical Nucleic Acid SNA (F-Bcl-2 ASO) Constructed by Bcl-2 Antisense Oligonucleotide of Skeleton Integrated Fluorouridine on the Drug-Resistant Liver Orthotopic Transplantation Tumor in Tumor-Bearing Nude Mice

In this example, the in vivo inhibitory effect of drug-resistant tumor proliferation of SNA (F-Bcl-2 ASO) was evaluated by using the model of drug-resistant liver orthotopic transplantation tumor in tumor-bearing nude mice.

In this example, PBS and equal equivalent fluorouridine, SNA(F-Bcl-2 ASO), SNA(F-scramble), and a mixture of spherical nucleic acid constructed by antisense oligonucleotide and fluorouridine SNA(Bcl-2 ASO)/F (wherein the equivalent concentration of fluorouridine for injection was 5 mg/kg bw) were injected through the tail vein, the magnetic resonance imaging and Siemens Inveon Research Workplace software were used to analyze and manually circle the tumor site of each layer by three-dimensional ordered subset expectation maximization (3D-OSEM), then the image was reconstructed and the tumor size was calculated. After the treatment, the tumor-bearing liver was dissected and taken out, and then the intuitive size of the drug-resistant liver orthotopic transplantation tumor was photographed and recorded.

The results is shown in FIG. 13, the initial size of the drug-resistant liver transplantation tumors in each treatment group is equivalent, the tumor size of the SNA(F-Bcl-2 ASO) treatment group is smaller than that of the PBS control group on the seventh and fifteenth days of the treatment, which shows a significant difference.

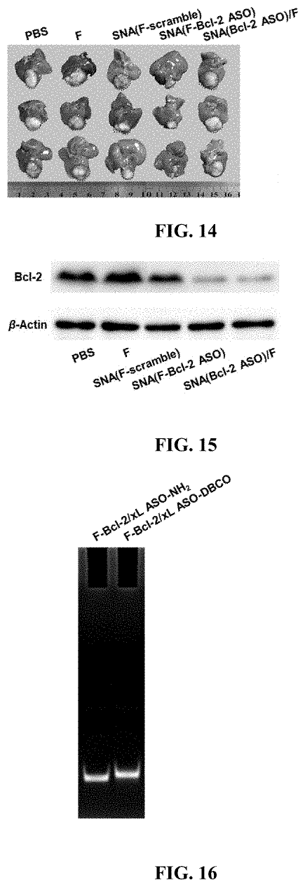

FIG. 14 shows a picture of the tumor-bearing liver taken out of the nude mice after the treatment, the white part in the picture is the drug-resistant liver orthotopic transplantation tumor. It can be seen from FIG. 13 and FIG. 14 that the tumor size in the SNA(F-Bcl-2 ASO) treatment group is the smallest after the treatment, while the tumor size in the remaining groups is only slightly smaller than that of the PBS control group, which means that SNA(F-Bcl-2 ASO) is the most effective drug of the above drugs.

3.5 Reversing the Tumor Drug Resistance by Spherical Nucleic Acid SNA (F-Bcl-2 ASO) Constructed by Bcl-2 Antisense Oligonucleotide of Skeleton Integrated Fluorouridine Through Gene Regulation

In order to verify whether the spherical nucleic acid SNA (F-Bcl-2 ASO) constructed by Bcl-2 antisense oligonucleotide of skeleton integrated fluorouridine could reverse tumor drug resistance through gene regulation, the tumor tissue was taken out after the treatment in this example, and the total protein was extracted quickly, the expression amount of drug-resistant protein in subcutaneous drug-resistant tumors was determined by western blot analysis.

The results of the determination are shown in FIG. 15, the Bcl-2 protein band in the treatment group of SNA(F-Bcl-2 ASO) is weaker than other groups, which indicates that SNA(F-Bcl-2 ASO) can down-regulate the expression amount of drug-resistant proteins in tumor-bearing nude mice. Therefore, SNA(F-Bcl-2 ASO) can show excellent gene therapy effects in animals and can effectively reverse the drug resistance of drug-resistant tumors.

### Example 4 a Spherical Nucleic Acid SNA (F-Bcl-2/xL ASO) Constructed by Bcl-2/xL Antisense Oligonucleotide of Skeleton Integrated Fluorouridine

4.1 Synthesis of the Bcl-2/xL Antisense Oligonucleotide of Skeleton Integrated Floxuridine Modified by DBCO

The Bcl-2/xL antisense oligonucleotide (F-Bcl-2/xL ASO-NH₂) of skeleton integrated floxuridine modified by amino was prepared by using the amino-modified controlled pore glass (NH₂-CPG) when preparing Bcl-2/xL antisense oligonucleotide of skeleton integrated floxuridine with a solid-phase synthesis method.

The above-mentioned antisense oligonucleotide sequence was added into a DMSO mixed solution containing 30% phosphate buffer, 200 equivalent of DBCO-NHS ester was added, and the mixture was reacted at room temperature for 24 hours to obtain a Bcl-2/xL antisense oligonucleotide (F-Bcl-2/xL ASO-DBCO) of skeleton integrated floxuridine modified by DBCO.

The above crude product was purified by multiple extractions with ethyl acetate, ethanol precipitation, and centrifugation.

Finally, the F-Bcl-2/xL ASO-DBCO was redissolved, and the DNA modification was characterized by gel electrophoresis. It can be seen from FIG. 16 that the redissolved products are presented as a single band after electrophoresis, and the products move slowly in gel electrophoresis compared with F-Bcl-2/xL ASO-NH₂, which proves the successful preparation of F-Bcl-2/xL ASO-DBCO.

4.2 Synthesis of Spherical Nucleic Acid SNA (F-Bcl-2/xL ASO) Constructed by Bcl-2/xL Antisense Oligonucleotide of Skeleton Integrated Fluorouridine

The polymer 1 (N₃-PEG-b-PCL₂₈) in example 3 and the Bcl-2/xL antisense oligonucleotide (F-Bcl-2/xL ASO-DBCO) of skeleton integrated floxuridine modified by DBCO were conjugated through classic click reaction.

The synthesis process was as follows: First, N₃-PEG-b-PCL₂₈ (200 nmol) was dissolved in 1.2 mL of DMSO solution, F-Bcl-2/xL ASO-DBCO (400 nmol) was dissolved in 30.0 μL of water, the two solutions were shaken at 58° C. for 48 h after mixing uniformly.

After the reaction, the reaction solution was placed in a dialysis bag with a molecular weight cut-off of 10 kDa for dialysis to remove DMSO. During the dialysis process, the N₃-PEG-b-PCL₂₈ and F-Bcl-2/xL ASO-DBCO conjugate would gradually assemble to form a spherical nucleic acid structure SNA (F-Bcl-2/xL ASO).

The excess antisense oligonucleotides that did not participate in the click reaction were removed by ultrafiltration with an ultrafiltration tube with a molecular weight cut-off of 100 kDa.

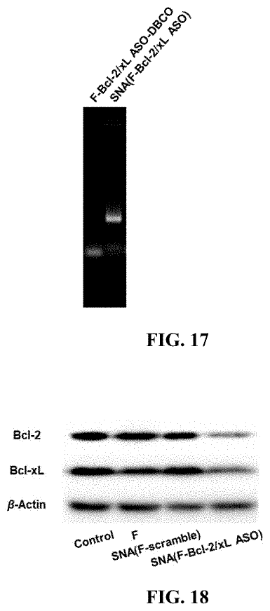

The purified SNA (F-Bcl-2/xL ASO) was characterized by agarose gel electrophoresis for its structure formation. The agarose gel concentration was 1%, the electrophoresis voltage was 90 V, and the gel imaging system was used for imaging after the electrophoresis was completed. The characterization results are shown in FIG. 17, the SNA (F-Bcl-2/xL ASO) band is located above the F-Bcl-2/xL ASO-DBCO band of the control group, which proves that the successful preparation of spherical nucleic acid SNA (F-Bcl-2/xL ASO) constructed by Bcl-2/xL antisense oligonucleotide of skeleton integrated fluorouridine.

4.3 Reversing the Tumor Drug Resistance by the Spherical Nucleic Acid SNA (F-Bcl-2/xL ASO) Constructed by Bcl-2/xL Antisense Oligonucleotide of Skeleton Integrated Fluorouridine Through Gene Regulation

The spherical nucleic acid SNA (F-Bcl-2/xL ASO) constructed by Bcl-2/xL antisense oligonucleotide of skeleton integrated fluorouridine were incubated with drug-resistant BEL-7402 cells for 10 hours, and then the incubation products were cultured for 48 hours after replacing by normal medium; while SNA(F-scramble) and F were used as control groups, which were incubated with cells under the same condition, wherein the equivalent concentration of F was 10 μM, and drug-resistant cells without any treatment were used as a negative control group.

The protein expression amount of the target protein Bcl-2 and protein Bcl-2/xL in the cells was determined by Western blot analysis after extracting the total protein. The results are shown in FIG. 18, which indicates that the SNA(F-Bcl-2/xL ASO) can significantly down-regulate the expression amount of drug-resistant Bcl-2 and Bcl-xL proteinsis after being incubated with drug-resistant cells BEL-7402, whereas the expression amount of Bcl-2 protein in the SNA(F-scramble) and F treatment groups have no significant difference compared with the blank control groups. Thus, the spherical nucleic acid (F-Bcl-2/xL ASO) constructed by Bcl-2/xL antisense oligonucleotide of skeleton integrated fluorouridine can reverse the drug resistance of drug-resistant tumors.

Compared with the prior art, the functional nucleic acid having nucleoside analog drug integrated into skeleton and the derivative thereof in the disclosure can efficiently enter cells, and the functional nucleic acid having nucleoside analog drug integrated into skeleton can be used to regulate genes; subsequently, the functional nucleic acid having nucleoside analog drug integrated into skeleton can be degraded by nuclease and release active ingredients of the nucleoside analog drug, thus playing a role in chemotherapy. Hence, the functional nucleic acid having nucleoside analog drug integrated into skeleton and the derivative thereof can simply and efficiently realize a combination therapy of gene therapy and chemotherapy, and a complex synthesis procedure can be avoided.

### Example 5 a PLK1 Small Interfering RNA of Skeleton Integrated Fluorouridine (F-siPLK1)

5.1 Synthesis of the PLK1 Small Interfering RNA of Skeleton Integrated Fluorouridine (F-siPLK1)

During in vitro enzymatic transcription, the nucleotide U in the siRNA was all replaced with the anti-tumor drug fluorouridine (F) in this example. Specifically,

T7 RNA polymerase Y639F was added to the transcription reaction solution for RNA synthesis, in which template DNA (1 μg), ATP (5 mM), CTP (5 mM), GTP (5 mM), 5-FdUTP (5 mM), DTT (10 mM)) and reaction buffer were contained, followed by incubation at 37° C. for 6 h. After the reaction, the product was purified by denatured polyacrylamide gel slices and recovered, and the target RNA segment was precipitated with ice ethanol at −20° C. The RNA was stored in −80° C. refrigerator to reserve after centrifugation and redissolution of RNA.

### Example 6 a Bel-2 Antisense Oligonucleotide (Gₑ-Bcl-2 ASO) of Skeleton Integrated Gemcitabine

6.1 Synthesis of the Bcl-2 Antisense Oligonucleotide (Ge-Bcl-2 ASO) of Skeleton Integrated Gemcitabine

The T nucleotides in the antisense oligonucleotides were all replaced with anti-tumor drugs gemcitabine (Gₑ) in this example during the solid-phase DNA synthesis. Specifically,

the phosphorous amide monomer and DNA phosphorous amide monomer of the gemcitabine drug were placed at the corresponding positions of the DNA solid-phase synthesizer, common controlled pore glass (CPG) were added to the reaction column, and the 5′-GₑAGGₑGTGGₑGGₑGₑATGₑGₑTTGₑGₑGₑATGₑGₑTGₑGₑTGₑGₑ-3′ sequence information was input, and catalytic, capping, oxidation and deprotection reagents were added, the Gₑ-Bcl-2 ASO sequence was obtained through ammonolysis, nitrogen blowing, separation and purification of preparative chromatographic, deprotection, and concentration after synthesizing the sequence containing gemcitabine.

The preferred embodiments disclosed above are only used to help explain the disclosure. The preferred embodiments do not describe all the details, and the disclosure is not limited to the specific embodiments. Obviously, many modifications and variations can be made according to the content of the description. These embodiments are selected and described in the description in order to explain the principles and practical applications, so that those skilled in the art can understand and utilize the disclosure well. The disclosure is limited only by the claims and their full scope and equivalents.

1. A functional nucleic acid having nucleoside analog drug integrated into skeleton, wherein the nucleoside analog drug is integrated into the skeleton of the functional nucleic acid by replacing natural nucleotides in the functional nucleic acid after chemical modifications:
wherein the ratio of gene drugs and chemotherapeutic drugs is able to be precisely controlled by adjusting the number of natural nucleotides replaced by the nucleoside analog drugs;
wherein the functional nucleic acid is selected from antisense oligonucleotides, small interfering RNA, messenger RNA, microRNA, long non-coding RNA, small hairpin RNA, guide RNA for gene editing, and circular RNA;
wherein the nucleoside analog drug is selected from purine analogs, guanosine analogs, cytidine analogs, adenosine analogs and uridine analogs.

2-6. (canceled)

7. The functional nucleic acid having nucleoside analog drug integrated into skeleton according to claim 1, wherein a method for preparing the functional nucleic acid having nucleoside analog drug integrated into skeleton is selected from the followings:
using a phosphoramidite monomer of the nucleoside analog drug, preparing a functional nucleic acid having nucleoside analog drug integrated into skeleton by solid-phase synthesis method; and
using a triphosphate monomer of the nucleoside analog drug, preparing a functional nucleic acid having nucleoside analog drug integrated into skeleton by in vitro enzyme transcription method or PCR amplification.

8. The functional nucleic acid having nucleoside analog drug integrated into skeleton according to claim 7, wherein the method comprising the following steps:
inputting the sequence of the functional nucleic acid to be synthesized into a solid-phase synthesizer, and preparing the functional nucleic acid having nucleoside analog drug integrated into skeleton on the surface of ordinary CPG or an amino-modified CPG;
reacting a mixed solution of RNA polymerase, template DNA, RNA nucleoside triphosphate monomer, triphosphate monomer of nucleoside analog drug, and reaction buffer at 37° C. to prepare the functional nucleic acid having nucleoside analog drug integrated into skeleton; and
reacting a mixed solution of DNA polymerase, template DNA, DNA primer, DNA nucleoside triphosphate monomer, triphosphate monomer of nucleoside analog drug, and reaction buffer at 37° C. to prepare the functional nucleic acid having nucleoside analog drug integrated into skeleton.

9. A derivative of the functional nucleic acid having nucleoside analog drug integrated into skeleton according to claim 1, wherein the derivative is selected from the followings:
a functional derivative that formed by combining the functional nucleic acid having nucleoside analog drug integrated into skeleton with a molecular targeting group;
a derivative and a self-assembled nanostructure thereof that obtained by modifying the functional nucleic acid having nucleoside analog drug integrated into skeleton with a polymer or a hydrophobic molecule;
a derivative of composite nanostructure that obtained by self-assembling the functional nucleic acid having nucleoside analog drug integrated into skeleton with a transfection reagent; and
a derivative of composite nanostructure that obtained by self-assembling the functional nucleic acid modified by a polymer or a hydrophobic molecule with a transfection reagent.

10. The derivative according to claim 9, wherein a method for preparing the derivative of the functional nucleic acid having nucleoside analog drug integrated into skeleton is selected from the followings:
using targeting molecules to modify the functional nucleic acid, obtaining the derivatives of the functional nucleic acid having nucleoside analog drug integrated into skeleton;
using polymers to modify the functional nucleic acid, obtaining the derivatives of the functional nucleic acid having nucleoside analog drug integrated into skeleton;
using hydrophobic molecules to modify the functional nucleic acid, obtaining the derivatives of the functional nucleic acid having nucleoside analog drug integrated into skeleton;
self-assembling the functional nucleic acid having nucleoside analog drug integrated into skeleton with a transfection reagent to obtain a derivative of composite nanostructure; and
self-assembling the functional nucleic acid having nucleoside analog drug integrated into skeleton or modified product thereof that modified by polymers or hydrophobic molecules with the transfection reagent to obtain a derivative of composite nanostructure.

11. The derivative of the functional nucleic acid having nucleoside analog drug integrated into skeleton according to claim 10, wherein the method using polymer modification comprises the following steps:
providing a degradable polymer with an azide group at the terminus synthesized by ring-opening polymerization, and then carrying out a copper-free catalytic click reaction between the degradable polymers and the functional nucleic acid having nucleoside analog drug integrated into skeleton modified by diphenyl-cyclooctyne, obtaining a conjugate of the degradable polymer-functional nucleic acid having nucleoside analog drug integrated into skeleton, as one of the derivatives of the functional nucleic acid having nucleoside analog drug integrated into skeleton.

12. A method for treating-diseases based on the combination of gene therapy and chemotherapy, wherein the method uses the functional nucleic acid having nucleoside analog drug integrated into skeleton according to claim 1 and/or derivatives thereof.

13. The derivative according to claim 9, wherein a method for preparing the functional nucleic acid having nucleoside analog drug integrated into skeleton is selected from the followings:
using a phosphoramidite monomer of the nucleoside analog drug, preparing a functional nucleic acid having nucleoside analog drug integrated into skeleton by solid-phase synthesis method; and
using a triphosphate monomer of the nucleoside analog drug, preparing a functional nucleic acid having nucleoside analog drug integrated into skeleton by in vitro enzyme transcription method or PCR amplification.

14. The derivative according to claim 9, wherein a method comprising the following steps:
inputting the sequence of the functional nucleic acid to be synthesized into a solid-phase synthesizer, and preparing the functional nucleic acid having nucleoside analog drug integrated into skeleton on a surface of ordinary CPG or an amino-modified CPG;
reacting a mixed solution of RNA polymerase, template DNA, RNA nucleoside triphosphate monomer, nucleoside analog drug triphosphate monomer, and reaction buffer at 37° C. to prepare the functional nucleic acid having nucleoside analog drug integrated into skeleton; and
reacting a mixed solution of RNA polymerase, template DNA, RNA primer, nucleoside analog drug triphosphate monomer, and reaction buffer at 37° C. to prepare the functional nucleic acid having nucleoside analog drug integrated into skeleton.

15. The derivative according to claim 13, wherein the method for preparing the derivative of the functional nucleic acid having nucleoside analog drug integrated into skeleton is selected from the followings:
using targeting molecules to modify the functional nucleic acid, obtaining the derivatives of the functional nucleic acid having nucleoside analog drug integrated into skeleton;
using polymers to modify the functional nucleic acid, obtaining the derivatives of the functional nucleic acid having nucleoside analog drug integrated into skeleton;
using hydrophobic molecules to modify the functional nucleic acid, obtaining the derivatives of the functional nucleic acid having nucleoside analog drug integrated into skeleton;
self-assembling the functional nucleic acid having nucleoside analog drug integrated into skeleton with a transfection reagent to obtain a composite nanostructure derivative; and
self-assembling the functional nucleic acid having nucleoside analog drug integrated into skeleton or modified product thereof that modified by polymers or hydrophobic molecules with the transfection reagent to obtain a derivative of composite nanostructure.

16. The derivative according to claim 14, wherein the method for preparing the derivative of the functional nucleic acid having nucleoside analog drug integrated into skeleton is selected from the followings:
using targeting molecules to modify the functional nucleic acid, obtaining the derivatives of the functional nucleic acid having nucleoside analog drug integrated into skeleton;
using polymers to modify the functional nucleic acid, obtaining the derivatives of the functional nucleic acid having nucleoside analog drug integrated into skeleton;
using hydrophobic molecules to modify the functional nucleic acid, obtaining the derivatives of the functional nucleic acid having nucleoside analog drug integrated into skeleton;
self-assembling the functional nucleic acid having nucleoside analog drug integrated into skeleton with a transfection reagent to obtain a composite nanostructure derivative; and
self-assembling the functional nucleic acid having nucleoside analog drug integrated into skeleton or modified product thereof that modified by polymers or hydrophobic molecules with the transfection reagent to obtain a derivative of composite nanostructure.

17. The derivative according to claim 15, wherein the method using polymer modification comprises the following steps:
providing a degradable polymer with an azide group at the terminus synthesized by ring-opening polymerization, and then carrying out a copper-free catalytic click reaction between the degradable polymers and the functional nucleic acid having nucleoside analog drug integrated into skeleton modified by diphenyl-cyclooctyne, obtaining a conjugate of the degradable polymer-functional nucleic acid having nucleoside analog drug integrated into skeleton, as one of the derivatives of the functional nucleic acid having nucleoside analog drug integrated into skeleton.

18. The derivative according to claim 16, wherein the method using polymer modification comprises the following steps:
providing a degradable polymer with an azide group at the terminus synthesized by ring-opening polymerization, and then carrying out a copper-free catalytic click reaction between the degradable polymers and the functional nucleic acid having nucleoside analog drug integrated into skeleton modified by diphenyl-cyclooctyne, obtaining a conjugate of the degradable polymer-functional nucleic acid having nucleoside analog drug integrated into skeleton, as one of the derivatives of the functional nucleic acid having nucleoside analog drug integrated into skeleton.

19. The method according to claim 12, wherein the method for preparing the functional nucleic acid having nucleoside analog drug integrated into skeleton is selected from the followings:
using a phosphoramidite monomer of the nucleoside analog drug, preparing a functional nucleic acid having nucleoside analog drug integrated into skeleton by solid-phase synthesis method; and
using a triphosphate monomer of the nucleoside analog drug, preparing a functional nucleic acid having nucleoside analog drug integrated into skeleton by in vitro enzyme transcription method or PCR amplification.

20. The method according to claim 12, wherein the method comprising the following steps:
inputting the sequence of the functional nucleic acid to be synthesized into a solid-phase synthesizer, and preparing the functional nucleic acid having nucleoside analog drug integrated into skeleton on a surface of ordinary CPG or an amino-modified CPG;
reacting a mixed solution of RNA polymerase, template DNA, RNA nucleoside triphosphate monomer, nucleoside analog drug triphosphate monomer, and reaction buffer at 37° C. to prepare the functional nucleic acid having nucleoside analog drug integrated into skeleton; and
reacting a mixed solution of RNA polymerase, template DNA, RNA primer, nucleoside analog drug triphosphate monomer, and reaction buffer at 37° C. to prepare the functional nucleic acid having nucleoside analog drug integrated into skeleton.

21. The method according to claim 12, wherein the derivative is selected from the followings:
a functional derivative that formed by combining the functional nucleic acid having nucleoside analog drug integrated into skeleton with a molecular targeting group;
a derivative and a self-assembled nanostructure of the derivative that obtained by modifying the functional nucleic acid having nucleoside analog drug integrated into skeleton with a polymer or a hydrophobic molecule:
a derivative of composite nanostructure that obtained by self-assembling the functional nucleic acid having nucleoside analog drug integrated into skeleton with a transfection reagent; and
a derivative of composite nanostructure that obtained by self-assembling a product with a transfection reagent, wherein the product is obtained by modifying the functional nucleic acid having nucleoside analog drug integrated into skeleton with a polymer or a hydrophobic molecule.

22. The method according to claim 12, wherein the method for preparing the derivative of the functional nucleic acid having nucleoside analog drug integrated into skeleton is selected from the followings:
using targeting molecules to modify the functional nucleic acid, obtaining the derivatives of the functional nucleic acid having nucleoside analog drug integrated into skeleton;
using polymers to modify the functional nucleic acid, obtaining the derivatives of the functional nucleic acid having nucleoside analog drug integrated into skeleton;
using hydrophobic molecules to modify the functional nucleic acid, obtaining the derivatives of the functional nucleic acid having nucleoside analog drug integrated into skeleton;
self-assembling the functional nucleic acid having nucleoside analog drug integrated into skeleton with a transfection reagent to obtain a composite nanostructure derivative; and
self-assembling the functional nucleic acid having nucleoside analog drug integrated into skeleton or modified product thereof that modified by polymers or hydrophobic molecules with the transfection reagent to obtain a derivative of composite nanostructure.

23. The method according to claim 12, wherein the method of modifying with polymers comprising the following steps:
providing a degradable polymer with an azide group at the terminus synthesized by ring-opening polymerization, and then carrying out a copper-free catalytic click reaction between the degradable polymers and the functional nucleic acid having nucleoside analog drug integrated into skeleton modified by diphenyl-cyclooctyne, obtaining a conjugate of the degradable polymer-functional nucleic acid having nucleoside analog drug integrated into skeleton, as one of the derivatives of the functional nucleic acid having nucleoside analog drug integrated into skeleton.
